(19) United States
(12) Patent Application Publication
(10) Pub. No.: US 2007/0032765 A1
(43) Pub. Date: Feb. 8, 2007

(54) Excrement treating apparatus

(76) Inventor: Toshiharu Honda, Nagasaki (JP)

Correspondence Address: WENDEROTH, LIND & PONACK, L.L.P., 2033 K STREET N. W., SUITE 800, WASHINGTON, DC, 20006-1021, US

(21) Appl. No.: 10/577,356

(22) PCT Filed: Oct. 29, 2004
(86) PCT No.: PCT/JP04/16134

§ 371 Date: Apr. 28, 2006

(30) Foreign Application Priority Data
Oct. 31, 2003 (JP) 2003-373309

(51) Int. Cl. A61F 5/44

(52) U.S. Cl. 604/347; 604/348

(57) ABSTRACT

An excrement treatment apparatus, wherein a hollow cup which is formed in such a shape that covers the private part and the anus of a human body having a hip part put on the upper side of a floor plate and in which at least the portion thereof in contact with the human body when the hollow cup abuts on the human body to cover the private part and the anus of the human body is formed of a soft elastic material is installed, in an upright state, contiguously with the upper side of the floor plate on which the hip part of the human body rests and in which at least the portion thereof in contact with the hip part of the human body put thereon is formed of the soft elastic material. An excrement discharge chamber having an excrement outflow space is formed inside the contiguously connected part of the floor plate with the hollow cup. A flushing means, a section/discharge means and a drying means are connected to the excrement discharge chamber and/or the hollow cup.

### BACKGROUND

1. Technical Field

The present invention relates to an excrement treatment apparatus that is developed for patients who lie in bed and always wear diapers because they cannot excrete and/or urinate by themselves or without the help of others, and cannot dispose of what they excreted and/or urinated by themselves or without the help of others. In particular, the present invention relates to the excrement treatment apparatus that is designed for use by a patient who is permanently bed-ridden, eliminating the need of using diapers and allowing the patient to change his or her posture with ease and to wash and dry the secrets such as the private part and anus of the human body with the apparatus being mounted on the body, thereby keeping the private part and/or anus clean at all times.

2. Description of the Prior Art

There is proposed a conventional excrement treatment apparatus that includes a diaper cup body surrounding the waist or hip portion of the human body, a flushing water delivery hose connected to the diaper cup body for delivering the flushing water into the diaper cup body, and an excrement suction hose connected to the diaper cup body for drawing the flushing water and excrement from the diaper cup body under the action of suction. In such excrement treatment apparatus, it is also proposed that the diaper cup body includes a joint in the form of bellows composed of a plurality of bent links connected with each other operatively and flexibly, and a front cover disposed on the tip end of the bellows joint. Examples of such excrement treatment apparatus are disclosed in the Japanese patent No. 3077083, the Japanese patent application now published for the public examination under No. 2000-79136 and the Japanese patent application now published for the public examination under No. 2003-24360.

The conventional excrement treatment apparatus described above is designed to be mounted on the waist or hip portion of a patient who lies in bed. This makes it difficult for the patient to change his or her posture with the apparatus being mounted on the human body or requires lots of labor for mounting it on the human body.

### SUMMARY OF THE INVENTION

The present invention solves the problems associated with the prior art described above, by providing an excrement treatment apparatus in which the cup that is provided for covering the secrets such as the private part and anus of a patient may be made of a soft elastic material, such as silicone rubber and artificial skin sheet, which is agreeable with the human body comfortably, and may be fitted on the waist or hip portion under the action of suction when it is being mounted to cover the secrets such as the private part and anus. As such, the patient can change his or her posture easily with the apparatus being mounted on the human body. In the excrement treatment apparatus according to the present invention, any means that would surround the waist or hip portion when it is mounted is eliminated.

Specifically, the excrement treatment apparatus proposed by the present invention may be characterized by the fact that a hollow cup which is formed in such a shape that covers the private part and the anus of a human body having a hip part put on the upper side of a floor plate and in which at least the portion thereof in contact with the human body when the hollow cup abuts on the human body to cover the private part and the anus of the human body is formed of a soft elastic material, wherein the hollow cup is installed, in an upright state, contiguously with the upper side of the floor plate on which the hip part of the human body rests and in which at least the portion thereof in contact with the hip part of the human body put thereon is formed of the soft elastic material. An excrement discharge chamber having an excrement outflow space is formed inside the part of the floor plate connected contiguously with the hollow cup. A flushing means, a section/discharge means and a drying means are connected to the discharge chamber and/or the hollow cup.

As one variation of the excrement treatment apparatus described above, it may further include a detecting device in the form of a sensor that is responsive to the presence of any excrement in the excrement discharge chamber, wherein the sensor may be mounted so that it can be exposed to the inner wall of the excrement discharge chamber or may be mounted so that it can be buried in the inner wall of the excrement discharge chamber.

As another variation of the excrement treatment apparatus, it may further include a detecting device in the form of a sensor that is responsive to the present of any excrement in the excrement discharge chamber or hollow cup, wherein the sensor may be mounted so that it can be exposed to the inner wall of the hollow cup or may be mounted so that it can be buried in the inner wall of the hollow cup.

In any of the excrement treatment apparatus and variations thereof, the hollow cup that is provided for abutting on the human body to cover the private part and anus or the excrement discharge chamber may internally be placed under the reduced pressure so that the hollow cup can be mounted on the human body watertightly under the action of suction.

In any of the excrement treatment apparatus and variations thereof, the flushing means may include a flushing water delivery pipe having one end thereof connected to the excrement discharge chamber and/or hollow cup and having the other end thereof connected to a hot water supply tank.

In any of the excrement treatment apparatus and variations thereof, the suction/excrement discharge means may include an excrement discharge pipe having one end thereof connected to the excrement discharge chamber and/or hollow cup and having the other end thereof connected to an excrement collecting tank having a suction generator provided therein.

In any of the excrement treatment apparatus and variations thereof, the drying means may include a hot air delivery pipe having one end thereof connected to the excrement discharge chamber and/or hollow cup and having the other end thereof connected to a hot air generator.

In this case, the drying means may further include a sterilizing/cleaning device that is capable of sterilize/cleaning the hot air supplied through the hot water delivery pipe into the excrement discharge chamber.

In any of the excrement treatment apparatus and variations thereof, it may further include a male genital guide tube that may be mounted removably inside the hollow cup.

In any of the excrement treatment apparatus and variations thereof, the sterilizing/cleaning device or discharged air processing device may be any device that is known in the relevant field as being capable of sterilizing/cleaning or deodorizing/sterilizing the discharged air. As an example of the known device that may be used to sterilize, clean or deodorize the air, there is an air sterilizing and deodorizing device that utilizes ozone or ultraviolet rays.

In any of the excrement treatment apparatus and variations thereof, the fact that the flushing means, the suction/discharge means and the drying means are connected to the excrement discharge chamber and/or the hollow cup should be understood to mean that the flushing means, the suction/excrement discharge means or the drying means may be connected to both or either of the excrement discharge chamber and hollow cup, and that some of the means such as, for example, the flushing means and suction/excrement discharge means may be connected to the excrement discharge chamber and the remaining means such as, for example, the drying means may be connected to the hollow cup.

Although the flushing means, suction/excrement discharge means and drying means may be connected with either of the excrement discharge member and hollow cup, it is sufficient that the respective outlets contiguous to those means and provided on the inner wall of the excrement discharge chamber or on the inner wall of the hollow cup have only to be located in the appropriate positions that enable those means to perform their respective functions.

For example, the outlet contiguous to the suction/excrement discharge means and provided on the inner wall of the excrement discharge chamber or on the inner wall of the hollow cup may be located on the lower portion of the inner wall of the excrement discharge chamber in such a manner that the suction/excrement discharge means can perform its function.

Similarly, the respective outlets contiguous to the flushing means and drying means and provided on the inner wall of the excrement discharge chamber or on the inner wall of the hollow cup may be located on the inner wall of the discharge chamber or on the inner wall of hollow cup so that those means can perform their respective functions.

The soft elastic materials that may be used in conjunction with the present invention include soft silicone, silicone rubber, artificial skin sheet and the like. As those materials are agreeable comfortably with the skin of the human body, they may be used on the portion of the floor plate that abuts on the hip part of the human body when the hip part of the human body rests on the upper side of the floor plate, and may be used on the part of the hollow cup that is brought into contact with the human body so that it can cover the private part and anus of the human body when the human body rests on the upper part of the floor plate. It may be appreciated from the preceding description, therefore, that the excrement treatment apparatus can provide a comfortable feeling for the human body.

As the portion of the hollow cup that may be brought into contact with the human body so that it can cover the private part and anus of the human body is made of any of the soft elastic materials mentioned above, there is no risk that the patient will have a rash on the skin when the skin contacts the material. If a flap is provided around the portion of the hollow cup in contact with the human body, the risk that the patient will have a rash on the skin can be avoided more effectively. In this case, the flap will also provide a comfortable feeling even if the human body contacts the hollow cup watertightly.

When the excrement treatment apparatus is to be mounted on the human body in accordance with the present invention, it can be done simply and easily. Then, the patient can change his or her posture easily, with the apparatus being mounted on the human body.

The secrets such as the private part and anus of the patient can be washed by the flushing water and then dried, keeping the secrets clean (sterilized) at all times.

In particular, the patient who is bed-ridden through an illness can keep himself or herself in good shape through rehabilitation, since he or she can change the posture easily with the apparatus being mounted on the body.

### BRIEF DESCRIPTION OF DRAWINGS

FIG. 1 is a perspective view of the excrement treatment apparatus according to the preferred embodiment of the present invention;

FIG. 2 is a flowchart diagram illustrating the sequence of the operations of the functional components of the apparatus; and

FIG. 3 is a block diagram illustrating the interconnections of the functional components of the apparatus.

### BRIEF DESCRIPTION OF NUMERAL

1 Hollow cup

2 Floor plate

3 Excrement discharge chamber

4 Flushing pipe

5 Hot air supply pipe

6 Excrement discharge pipe

7 Bowel movement sensor

8 Controller

9 Narrow flap

10 Excrement treatment apparatus

13 Suction generator

14 Motor

15 Air discharge pump

16 Air discharge pipe

17 Excrement collecting tank

18 Suction hose

21, 23, 24 Valve

22 Hot water supply pipe

26 Air supply pipe

27 Hot water supply tank

### BEST MODE OF EMBODYING THE INVENTION

It may be appreciated from the preceding description that the excrement treatment apparatus according to the present invention provides a comfortable-feeling for the human body when it is mounted on the body, can fit any shape of the human body and permits the patient to change his or her posture with the apparatus being mounted on the body Particularly, the excrement treatment apparatus is improved in such a manner that it permits the user excrete or urinate easily when the user is a patient who cannot excrete or urinate by himself or herself without relying upon some helpers and that the flushing and drying operations can be performed automatically.

The following describes the functions that may be provided by the excrement treatment apparatus of the present invention by showing a particular embodiment thereof.

The portion of the excrement treatment apparatus that is brought into contact with the human body may be formed by using soft elastic materials such as soft silicone, silicone rubber or artificial skin that are made to be agreeable with the human skin. Specifically, at least the portion of the upper side of the floor plate on which the hip part of the human body may be placed and which is brought into contact with the hip part of the human body is formed of soft silicone, and the portion of the hollow cup having the shape that covers the private part and the anus of the human body and which may be brought into contact with the human body when the hip part of the human body is placed on the upper side of the floor plate may also be formed of soft silicone.

In accordance with the excrement treatment apparatus of the present invention, the hollow cup may be formed so that it can be suited to the average physical human body shape. If it is available in two different sizes, that is, if one is provided for the relatively large physical human body and the other is provided for the relatively small physical human body, the two different hollow cups can cover all types or sizes of human bodies.

As the hollow cup covers the private part and anus of the human body, it may be provided so that it can fit the private part and anus of the user or patient. If it is supposed, for example, that one person is 180 cm tall and the other person is 160 cm tall, there is no substantial difference in the distance between the private part and anus for those two different persons. Thus, if two different hollow cups are provided, they can satisfy the needs of all types of users.

Optionally, the excrement treatment apparatus may include a removable male genital guide tube that is only used by males. If this guide tube is removed, the apparatus may also be used by females.

Preferably, the portion of the male genital guide tube that is brought into contact with the human body may be formed of soft silicone, silicone rubber or artificial skin that is prepared so that it can be agreeable with the skin. The reasons have been described above.

The excrement treatment apparatus has been realized as the results of the research and development that have been made on the materials mentioned above, and provides a comfortable feeling for the patient who is bed-ridden, permitting the patient to change his or her posture easily and simply as the apparatus is mounted on the body at all times. It is as light as the ordinary diaper and has the function to permit the patient to excrete and urinate with the apparatus being mounted on the body.

The opening of the hollow cup has a flap around the entire circumference thereof that makes the user feel comfortable upon contacting it, although the contact is made watertight. A soft cloth may also be attached to the flap when it is actually used.

The flushing water supply pipe of the flushing means may be connected to the hot water supply tank through the flushing pipe and the like, and the hot water may be jetted as flushing water from the hot water supply tank into the hollow cup. All parts of the human body covered by the hollow cup may be cleaned by the flushing water.

The flushing time is variable, and may be set to any desired time, depending upon how the hollow cup is polluted.

The hot air supply pipe of the drying means may be connected to the hot air generator through the drying pipe, and the hot air may be jetted into the hollow cup where all parts of the human body just cleaned by the flushing water may be dried readily.

In the excrement treatment apparatus according to the present invention, the discharge pipe of the suction/excrement discharge means may be connected to the excrement collecting tank in which the suction generator is provided, and the excrement together with the accompanying odor can be drawn from the hollow cup under the forced suction and collected into the excrement collecting tank.

This forced suction reduces the pressure inside the hollow cup, introducing the air from the outside into the hollow cup. Thus, no leak will occur even if there is a slight gap between the hollow cup and human body that might be caused if the contact pressure between the hollow cup and human body is insufficient. This forced suction acts as the air curtain that shuts the hollow cup off the outside, preventing any odor from flowing from inside the hollow cup toward the outside. Thus, there is no risk that any odor, excrements and flushing water inside the hollow cup and excrement discharge chamber might leak toward the outside.

In the respects described above, the excrement treatment apparatus according to the present invention has the advantage over the prior art excrement treatment apparatus that has been popular among the users and mounted by those users. For example, when a band or similar device is used to mount the excrement treatment apparatus on the human body, it may be fastened too tightly or too loosely. It is therefore difficult to mount it in the best way. Otherwise, some experience or skill is required to mount it in the best way.

In accordance with the present invention, the hollow cup can be kept sufficiently watertight by simply placing the floor plate beneath the hip part of the human body, bringing the hollow cup into contact with the human body so that it can cover the private part and anus of the human body and reducing the pressure to produce the suction inside the hollow cup, and can also be kept airtight so that the odor cannot escape from within the hollow cup.

Even if the band is used in the present invention, there is no need of the hollow cup being bound by the band. Rather, the band can be fastened not tightly.

The excrement treatment apparatus of the present invention may further include a pressure balance pipe through which the pressurized air can be delivered into the hollow cup and into the excrement discharge chamber. In this way, the suction pressure cannot be controlled automatically so that no excessive pressure can be exerted.

The above object can be achieved by considering the size (diameter) of the pressure balance pipe, and can be attained more reliably by interposing valves that open and close automatically under the pressure differential. Thus, the pressure within the hollow cup and excrement discharge chamber can be adjusted more accurately.

In the excrement treatment apparatus of the present invention, sensors are provided in their respective positions for detecting the presence of any excrement such as bowel movement and urination. In response, the excrement may automatically be discharged (suction and removal), flushed and dried. Thus, the flushing and drying operation may occur in the adequate manner.

When the flushing and drying occur as described above, the flushing water and hot water may be sterilized. The dry sterilization may occur as required. This can remove any odor produced by the patient in bed.

The start-up and stop of the excrement treatment apparatus may be performed in two modes, that is, the automatic mode using sensors and the manual mode by remote control or button. Those two modes may be switched as required.

Embodiment

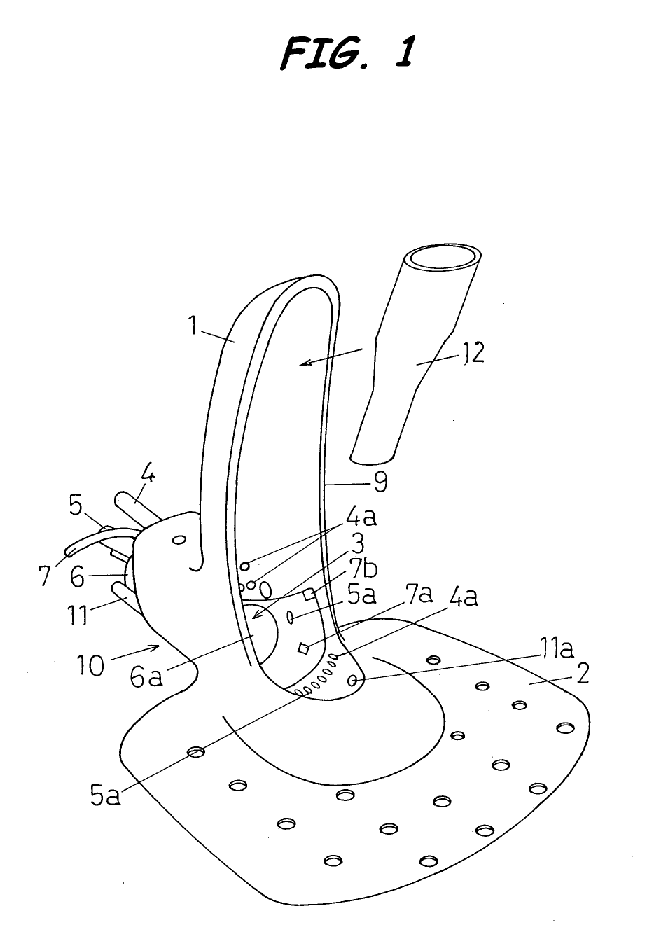

One particular embodiment of the excrement treatment apparatus 10 of the present invention and some variations thereof will be described below by referring to FIG. 1.

A hollow cup 1 is made of soft silicone rubber, and its bottom portion is provided contiguously to one side of a floor plate 2 made of soft silicone rubber on which the hip part of the human body may rest. Inside the region where the bottom portion of the hollow cup 1 and the one side of the floor plate 2 are contiguous to each other, there is a cylindrical excrement discharge chamber 3 having an excrement outflow space (whose capacity is approximately 1000 cm³) is provided.

The excrement discharge chamber 3 may also be made of soft silicone rubber.

Only the portion of the floor plate 2 that will be brought into contact with the hip part of the human body when the hip part rests on the upper side of the floor plate 2 may be made of soft silicone rubber.

Similarly, only the portion of the hollow cup 1 that will be brought into contact with the human body when the hollow cup 1 is made to abut on the human body so that it can cover the private part and anus of the human body when the hip part of the human body rests on the upper side of the floor plate 2 may be made of soft silicone rubber.

A flushing pipe 4, a hot air supply pipe 5 and an excrement discharge pipe 6 are connected to the outside wall of the excrement discharge chamber 3, respectively. Those pipes are open in their respective positions inside the excrement discharge chamber 3. For example, the flushing pipe 4 has flushing jet outlets 4a, the hot air delivery pipe 5 has hot air outlets 5a and the excrement discharge pipe 6 has an outlet 6a.

The excrement treatment apparatus 10 may further include a detecting device in the form of a sensor that is responsive to the presence of any excrement within the excrement discharge chamber 3, in which the sensor may be exposed to the inner wall of the excrement discharge chamber 3 or may be buried in the inner wall of the excrement discharge chamber 3.

Alternatively, the excrement treatment apparatus 10 may further include a detecting device in the form of a sensor that is responsive to the presence of any excrement within the hollow cup 1, in which the sensor may be exposed to the inner wall of the hollow cup 1 or may be buried in the inner wall of the hollow cup 1.

Both of the excrement discharge chamber 3 and hollow cup 1 may include the respective detecting device in the form of the sensor described above in each case.

In the embodiment shown in FIG. 1, a bowel movement sensor 7a and a urine sensor 7b are provided on the wall of the excrement discharge chamber 3, each of which is connected to one end of a sensor 7. The bowel movement sensor 7a may be responsive to the presence of any bowel movement, and the urine sensor 7b may be responsive to the presence of any urine. Both of the sensors 7a and 7b may provide electrical signals.

In addition, a temperature sensor that is connected to one end of the sensor 7 may be provided on the wall of the excrement discharge chamber 3. Similarly to the bowel movement sensor 7a and urine sensor 7b, this temperature sensor may be buried in the wall of the excrement discharge chamber 3 and/or hollow cup 1.

The other end of the sensor 7 is connected to a controller 8 that provides ON and OFF command signals for enabling and disabling a flushing means, a suction/excrement discharge means and a drying means which will be described later.

Within the excrement discharge chamber 3, there is a pressure balance pipe 11 that passes through the hollow cup 1 and balances the pressures within the excrement discharge chamber 3 and within the hollow cup 1 that is mounted watertightly on the human body. In FIG. 1, reference numeral 11a refers to the opening.

The hollow cup 1 has the shape that covers the private part and anus of a patient in bed, and has the arc shape in cross section such that it is rising like an arc toward the top from the floor plate 2 as shown in FIG. 1. The opening of the hollow cup 1 (which abuts on the human body) has a narrow flap 9 along the entire circumference thereof.

In FIG. 1, reference numeral 12 denotes a male genital guide tube that may be mounted removably inside the hollow cup 1.

When the excrement treatment apparatus 10 is used by a patient lying on his or her back, the patient may put his hip part on the floor plate 2 so that his or her private part and anus can be covered by the hollow cup 1.

The excrement treatment apparatus 10 may be mounted on the patient lying on his or her back by helping the patient spread his or her legs out, moving the apparatus 10 from the lower half side of the human body toward the upper half side, and sliding the floor plate 2 beneath the hip part of the human body so that the hollow cup 1 can cover the private part and anus of the patient. Then, the excrement treatment apparatus 10 may be mounted watertightly on the human body by reducing the pressures within the hollow cup 1 and within the excrement discharge chamber 3 to produce the suction therein, when the hollow cup 1 abuts on the hip part of the human body for covering the private part and anus of the patient

In this state, the angle at which the hollow cup 1 is to be mounted with regard to the floor sheet 2 may be determined so that the opening of the hollow cup 1 can cover the private part and anus of the patient.

In the excrement treatment apparatus, a belt may be used as required so that the hollow cup 1 can be maintained to be opposite the secrets (the private part and anus) of the patient. As the hollow cup 1 is always mounted on the patient, this avoids that the secrets of the patient and the hollow cup 1 will slide relative to each other as the patient is moving, thereby ensuring that the patient can excrete with ease.

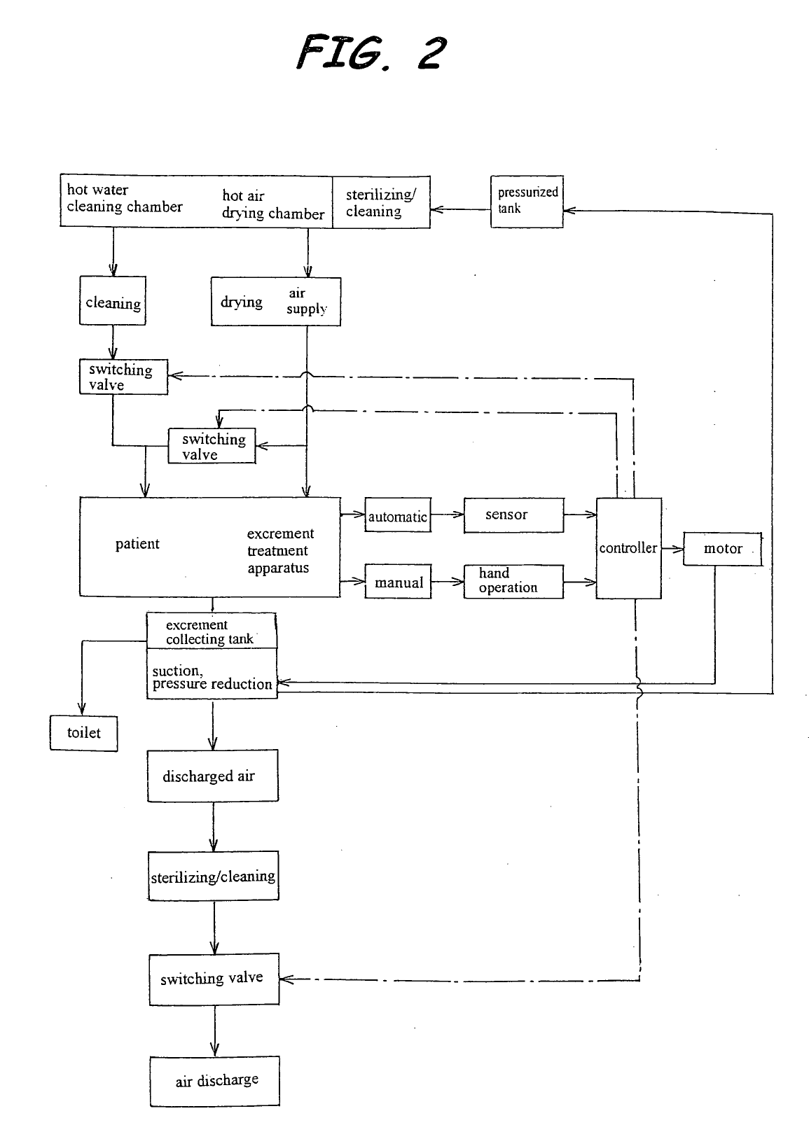

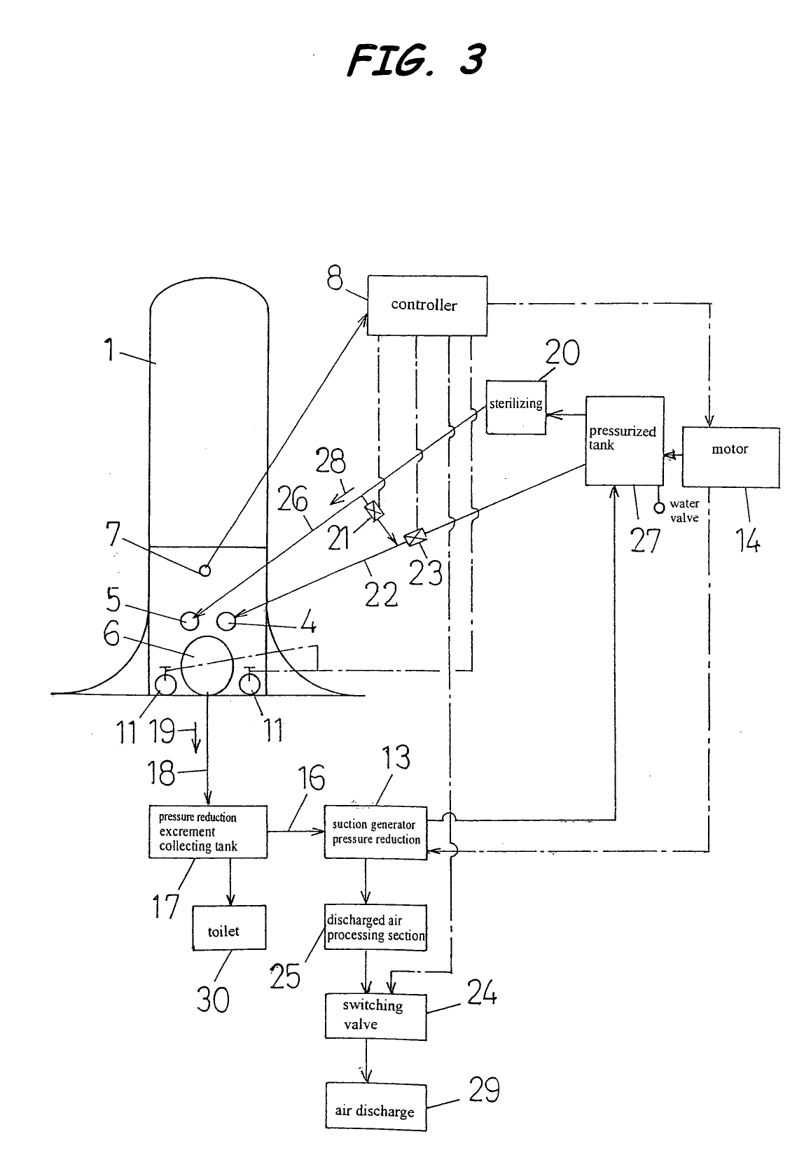

The operation of the excrement treatment apparatus is now described in accordance with the embodiment thereof shown in FIGS. 2 and 3. When the sensor 7a or sensor 7b detects the presence of any excrement or urination, it provides signals that are delivered through the sensor 7 to the controller 8.

In response, the controller 8 provides a command that enables the motor 14 of the suction generator 13 to start, reducing the pressure inside the excrement collecting tank 17 through the air discharge pipe 16.

When the pressure within the excrement collecting tank 17 is reduced, it causes the air to be discharged through the suction hose 18 and excrement discharge pipe 6, causing the pressures within the hollow cup 1 and within the excrement discharge chamber 3 to be reduced. Then, the excrement is drawn by the suction introduced from the suction pipe 19 and is collected through the excrement discharge pipe 6 into the excrement collecting tank 17.

When all excrement has been collected into the excrement collecting tank 17 under the suction introduced for a certain time (for example, one minute), the controller 8 provides command signals that open the valve 23 and close the valve 21.

The air discharged through the air discharge pipe 16 increases the pressure in the hot water supply tank 27, causing the hot water to be delivered from the water supply tank 27 through the hot water supply pipe 22 and flushing pipe 4 into the excrement discharge chamber 3 for flushing.

When the flushing is completed in the hollow cup 1 and in the excrement discharge chamber 3 after a certain time (for example, one minute), the valve 23 is closed and the valve 21 is opened, stopping the supply of the hot water. The hot water supply tank 27 is controlled automatically so that it can be warmed up to a certain temperature, e.g., 37° C. when it drops below the certain temperature.

In addition to discharging the excrement under the suction through the excrement discharge pipe 6 as described, the pressurized air, the hot water and the dry air may be jetted through the outlet 11a of the pressure balance pipe 11, through the flushing water jet outlet 4a and through the hot air jet outlet 5a, respectively, so that the excrement remaining within the discharge chamber 3 can be discharged more powerfully and forcedly through the discharge outlet 6a and then through the excrement discharge pipe 6. This is coupled with the suction through the excrement discharge pipe 6, and can discharge the excrement completely from the excrement discharge chamber 3, cleaning up the hollow cup 1 and excrement discharge chamber 3 and keeping them clean.

Then, the controller 8 provides a command signal that causes the hot air to be delivered through the blowing pipe 26 and hot air delivery pipe 5, as shown by an arrow 28, for drying the hollow cup 1 and excrement discharge chamber 3. When the hollow cup 1 and excrement discharge chamber 3 have completely been dried, a temperature sensor (not shown) responds through the sensor 7, stopping the motor 14. This concludes the sequence of the operations described so far.

In the preceding description, the air from the air discharge pump 15 may pass through the discharged air processing section 25 where it is deodorized and sterilized, and may then go out through the switching valve 24. For a patient lying in bed for a long time, any odor coming from the patient can thus be removed from the room in which the patient stays.

In the preceding description, the air from the suction generator 13 may pass through the discharged air processing section 15 where it is cleaned, from which the air may then go out through the switching valve 24 into the atmosphere. The air from the suction generator 13 may pass through the pressurizing tank 27, from which the air may pass through the sterilizing/cleaning section 20. Thus, the hollow cup 1 and excrement discharge chamber 3 can be sterilized and dried by the air from the sterilizing/drying section 20. Finally, the excrement collecting tank 17 may be detached, from which the contents may be discarded into the toilet 30.

The deodoring/sterilizing may be implemented by any known device that utilizes ozone or ultraviolet rays, and the air sterilizing, cleaning and deordoring section may be implemented by any known air sterilizing, cleaning and deordoring device.

1. An excrement treatment apparatus, including:
a hollow cup formed in such a shape that covers the private part and the anus of a human body having a hip part put on the upper side of a floor plate and in which at least the portion thereof brought into contact with the human body when the hollow cup abuts on the human body to cover the private part and the anus of the human body is formed of a soft elastic material, wherein the hollow cup is installed, in an upright state, contiguously with the upper side of the floor plate on which the hip part of the human body rests and in which at least the portion thereof in contact with the hip part of the human body put thereon is formed of the soft elastic material;
an excrement discharge chamber having an excrement outflow space and formed inside the part of the floor plate connected contiguously with the hollow cup; and
a flushing means, a section/excrement discharge means and a drying means each being connected to the excrement discharge chamber and/or the hollow cup.

2. The excrement treatment apparatus as defined in claim 1, further including a detecting device in the form of a sensor that is responsive to the presence of any excrement in the excrement discharge chamber, wherein the sensor may be mounted so that it can be exposed to the inner wall of the discharge chamber or may be mounted so that it can be buried in the inner wall of the discharge chamber.

3. The excrement treatment apparatus as defined in claim 1, further including a detecting means in the form of a sensor that is responsive to the presence of any excrement in the excrement discharge chamber or hollow cup, wherein the sensor may be mounted so that it can be exposed to the inner wall of the hollow cup or may be mounted so that it can be buried in the inner wall of the hollow cup.

4. The excrement treatment apparatus as defined in claim 1, wherein the hollow cup that is provided to abut on the human body to cover the private part and anus of the human body or the excrement discharge chamber may internally be placed under the reduced pressure so that the hollow cup can be mounted on the human body watertightly under the action of suction.

5. The excrement treatment apparatus as defined in claim 1, wherein the flushing means includes a flushing water supply pipe having one end thereof connected to the excrement discharge chamber and/or hollow cup and having the other end thereof connected to a hot water supply tank.

6. The excrement treatment apparatus as defined in claim 1, wherein the suction/excrement discharge means includes an excrement discharge pipe having one end thereof connected to the excrement discharge chamber and/or hollow cup and having the other end thereof connected to an excrement collecting tank having a suction generator provided therein.

7. The excrement treatment apparatus as defined in claim 1, wherein the drying means includes a hot air delivery pipe having one end thereof connected to the excrement discharge chamber and/or hollow cup and having the other end thereof connected to a hot air generator.

8. The excrement treatment apparatus as defined in claim 1, further including a male genital guide tube removably mounted inside the hollow cup.

9. The excrement treatment apparatus as defined in claim 1, further including a deodorizing/sterilizing means for deodorizing/sterilizing the discharged air drawn under the suction from the excrement discharge chamber through the discharge air pipe of the suction/excrement discharge means.

10. The excrement treatment apparatus as defined in claim 7, wherein the drying means further includes a sterilizing/cleaning device that is capable of sterilize/cleaning the hot air supplied through the hot air delivery pipe into the excrement discharge chamber.
